(19) United States
(12) Patent Application Publication
(10) Pub. No.: US 2024/0165035 A1
(43) Pub. Date: May 23, 2024

(54) COATED SOLID PHARMACEUTICAL PREPARATION

(71) Applicant: SSP CO., LTD., Tokyo (JP)

(72) Inventors: Yasuhisa ISHIKAWA, Chiba (JP); Gento KODAKA, Chiba (JP); Makoto UMINO, Chiba (JP); Yoichi ONUKI, Chiba (JP); Minoru OKADA, Chiba (JP)

(21) Appl. No.: 18/282,876

(22) PCT Filed: Mar. 18, 2022
(86) PCT No.: PCT/JP2022/012853

§ 371 Date: Sep. 19, 2023

(30) Foreign Application Priority Data
Mar. 19, 2021 (JP) 2021-045938

(51) Int. Cl. A61K 9/28; A61K 9/20

(52) U.S. Cl. CPC A61K 9/2846; A61K 9/2013; A61K 9/2054; A61K 9/2813

(57) ABSTRACT

Provided is a coated solid pharmaceutical preparation which not only makes it difficult to perceive the unpleasant taste and/or unpleasant odor of pharmacologically active substances when taken but also does not easily discolor over time. In the coated solid pharmaceutical preparation, a solid pharmaceutical preparation containing a pharmacologically active substance is coated with a film containing a film-forming polymer and a flaky substance.

### FIELD OF INVENTION

The present invention relates to a coated solid pharmaceutical preparation.

### BACKGROUND OF INVENTION

Conventionally, solid pharmaceutical preparations such as tablets, capsules, and granules are coated with polymers, sugar coatings, or the like, for reducing unpleasant taste and unpleasant odor when taken, improving the stability (for example, for preventing discoloration and odor over time or preventing whiskers or fogging of packaging containers due to sublimation), or preventing the direct contact with pharmacologically active substances.

In particular, a method of coating solid pharmaceutical preparations such as tablets, capsules, and granules with a film-forming polymer is often used since not only the required time for production is much shorter than that for sugar coatings and the production operation is also easy, but also various functions such as solubility in water, gastric solubility, enteric solubility, sustained release, and the like can be added depending on the types of polymers.

However, since the film formed by such a film-forming polymer is inferior in denseness as compared to sugar coatings, various ideas have been made.

For example, in order to shield oxygen or the odor of tablets, it is proposed to coat tablets with a film coating composition containing polyvinyl alcohol and 50 to 86 mass % of talc with respect to the solid content (Patent Literature 1: JPA-2006-188490). Further, in order to reduce the cysteine odor, it has been proposed to apply a coating film containing a partially saponified product of polyvinyl alcohol to a solid preparation containing L-cysteine or a salt thereof (Patent Literature 2: JPA-2008-201711 A).

Further, in order to suppress the discoloration or odor, it has been proposed to apply a coating film with a film thickness of 60 μm or more containing a polyvinyl alcohol copolymer to an uncoated tablet containing tranexamic acid, ascorbic acid, and L-cysteine (Patent Literature 3: WO-A-2011/049093).

Other than above, there have been reported a coating composition for solid preparations containing a drug unstable in water, the composition containing hydroxypropylcellulose, talc, propylene glycol, and polyethylene glycol at a specific compounding ratio (Patent Literature 4: JP-A-2007-001873), and a coating agent for solid preparations containing polyvinyl alcohol and bentonite or magnesium aluminum silicate (Patent Literature 5: WO-A-2010/074223).

However, these films could not sufficiently reduce the unpleasant taste and/or unpleasant odor of pharmacologically active substances. Further, it may have required a time for production due to an increase in the amount of coating.

Further, there have been problems that it is applicable only to tablets in a specific shape, a complicated and advanced technology, a special spray nozzle, or the like is required, the cost is high, and production with stable quality is difficult. Further, dissolution properties or disintegration properties may change due to excessively rapid or slow dissolution or disintegration of the coated solid pharmaceutical preparation, and the stability or appearance of the pharmacologically active substance may be affected by the deficiency or the like in the production process, so that the effect of the pharmacologically active substance could not be fully exerted.

### CITATION LIST

### Patent Literature

[PTL 1] JP-A-2006-188490
[PTL 2] JP-A-2008-201711
[PTL 3] WO-A-2011/049093
[PTL 4] JP-A-2007-001873
[PTL 5] WO-A-2010/074223

### SUMMARY OF INVENTION

### Technical Problem

The object of the present invention is to provide a coated solid pharmaceutical preparation which not only makes it difficult to perceive the unpleasant taste and/or unpleasant odor of pharmacologically active substances when taken and which also does not easily discolor over time.

### Solution to Problem

As a result of diligent studies in order to solve the above problems, the inventors found that application of a film containing a flaky substance together with a film-forming polymer to a solid pharmaceutical preparation containing a pharmacologically active substance not only makes it difficult to perceive the unpleasant taste and/or unpleasant odor of the pharmacologically active substance when taken, but also does not easily cause discoloration over time during storage of the preparation, thereby accomplishing the present invention.

That is, the disclosure provides a coated solid pharmaceutical preparation in which a solid pharmaceutical preparation comprising a pharmacologically active substance is coated with a film comprising a film-forming polymer and a flaky substance.

Further, the disclosure provides a method for suppressing unpleasant taste and/or unpleasant odor derived from a pharmacologically active substance when a solid pharmaceutical preparation comprising the pharmacologically active substance is taken, the method comprising applying a film comprising a film-forming polymer and a flaky substance to the solid pharmaceutical preparation.

Further, the disclosure provides a method for suppressing discoloration over time when a solid pharmaceutical preparation comprising a pharmacologically active substance is stored, the method comprising applying a film comprising a film-forming polymer and a flaky substance to the solid pharmaceutical preparation.

### Advantageous Effects of Invention

The coated solid pharmaceutical preparation of the disclosure not only makes it difficult to perceive the unpleasant taste and/or unpleasant odor of pharmacologically active substances when taken but also does not easily discolor over time during storage of the preparation.

### DETAILED DESCRIPTION OF INVENTION

<Coated Solid Pharmaceutical Preparation>

In the coated solid pharmaceutical preparation of the disclosure, a solid pharmaceutical preparation comprising a pharmacologically active substance is coated with a film comprising a film-forming polymer and a flaky substance (this film will be hereinafter referred to as “specific film”).

The pharmacologically active substance used in the coated solid pharmaceutical preparation of the disclosure is not specifically limited but is preferably a pharmacologically active substance which generally tends to cause unpleasant taste and/or unpleasant odor such as bitterness or strong irritation to be perceived when formed as a solid preparation, or which can generally cause problems in stability such as the unpleasant odor or discoloration over time when formed as a solid preparation. Even if such a pharmacologically active substance is used, the present invention can suppress the unpleasant taste and/or unpleasant odor when the pharmacologically active substance is taken and problems in stability such as the unpleasant odor or discoloration over time.

Examples of the pharmacologically active substance which tends to cause unpleasant taste include ascorbic acid, aspirin, acetaminophen, allylisopropylacetylurea, alprenolol, isoaminile, ibuprofen, etilefrine, ethenzamide, epinastine, ephedrines (such as pseudoephedrine and methylephedrine), erythromycin, caffeine, carbetapentane, quinine, chlordiazepoxide, chlorpheniramine, chlorpromazine, codeine, diazepam, digitoxin, dihydrocodeine, diphenhydramine, diphenhydramine hydrochloride, cimetidine, dimemorfan, dextromethorphan hydrogen bromide, diltiazem, cefaclor, celecoxib, talampicillin, thiamine, theophylline, delapril, tranexamic acid, noscapine, bacampicillin, Ca pantothenate, pirenzepine, pirenzepine hydrochloride, fexofenadine, pheniramine, fursultiamine, propranolol, promethazine, meclofenoxate, metamizole, meloxicam, loxoprofen, and loperamide.

Among these, ascorbic acid, aspirin, acetaminophen, ibuprofen, ethenzamide, epinastine, ephedrines, caffeine, chlorpheniramine, diphenhydramine, thiamine, theophylline, tranexamic acid, noscapine, Ca pantothenate, pirenzepine hydrochloride, fexofenadine, metamizole and meloxicam are preferable.

Examples of the pharmacologically active substance which tends to cause unpleasant odor when taken or over time include ibuprofen, glucosamine, chondroitin sulfate, Na chondroitin sulfate, hyaluronic acid, and methylmethionine sulfonium chloride, other than Chinese herbal extracts such as Kakkonto, Kufugedokuto, Kyouseihatekiganryou, Shosaikoto, Shoseiryuto, Sansonito, Jumihaidokuto, and Saikokeishito; crude drugs or biological extracts such as aloe, fennel, turmeric, valerian, licorice, kikuka, cinnamon, oriental bezoar, peony, ginger, earthworm, jujube, passionflower, ginseng, garlic, hop, and ephedra; amino acids such as isoleucine, cysteine, valine, methionine, and leucine; vitamin B1 and derivatives thereof; the vitamin C group including ascorbic acid and erythorbic acid; and vitamin E.

Among these, ascorbic acid, vitamin B1 and derivatives thereof, ibuprofen, Chinese herbal extracts, chondroitin sulfate, Na chondroitin sulfate, cysteine, methionine, crude drug extracts, and vitamin C are preferable.

Among the pharmacologically active substance which tends to cause unpleasant taste and the pharmacologically active substance which tends to cause unpleasant odor when taken or over time, ascorbic acid, aspirin, acetaminophen, ibuprofen, ethenzamide, epinastine, ephedrines, caffeine, Chinese herbal extracts, chlorpheniramine, chondroitin sulfate, Na chondroitin sulfate, cysteine, methionine, diphenhydramine, crude drug extracts, theophylline, tranexamic acid, noscapine, Ca pantothenate, vitamin B1 and derivatives thereof, vitamin C, pirenzepine hydrochloride, fexofenadine, and meloxicam are preferable.

Further, in the disclosure, at least one or more pharmacologically active substances selected from the group consisting of aspirin, acetaminophen, ibuprofen, ethenzamide, epinastine, ephedrines, caffeine, Chinese herbal extracts, chlorpheniramine, chondroitin sulfate, Na chondroitin sulfate, cysteine, diphenhydramine, crude drug extracts, theophylline, tranexamic acid, noscapine, Ca pantothenate, vitamin C, and pirenzepine hydrochloride are preferably contained, at least one or more pharmacologically active substances selected from the group consisting of aspirin, ibuprofen, ephedrines, epinastine, chondroitin sulfate, Na chondroitin sulfate, cysteine, diphenhydramine, tranexamic acid, Ca pantothenate, vitamin C, and pirenzepine hydrochloride are more preferably contained, at least one or more pharmacologically active substances selected from the group consisting of ibuprofen and cysteine are more preferably contained, and at least cysteine is particularly preferably contained. In addition, in specific embodiments of the disclosure, the pharmaceutically active substance is ascorbic acid (Vitamin C, aspirin, acetaminophen, ibuprofen, metamizole, caffeine, chlorpheniramine, diphenhydramine hydrochloride, Vitamin B1 and derivatives thereof. Ascorbic acid (Vitamin g, aspirin, acetaminophen, ibuprofen, metamizole, caffeine, chlorpheniramine, diphenhydramine hydrochloride, Vitamin B1 and derivatives may be present in coated-solid preparation in any of the amounts shown in Embodiments A-C in the Table below.

TABLE 1

| | Embodiment A | | Embodiment B | | Embodiment C |
|---|---|---|---|---|---|
| | Amount of | | Amount of | | Amount of |
| | active | | active | | active |
| Active Substance | substance | | substance | | substance |
| Ascorbic acid | | | | | 666 mg |
| (Vitamin C) | | | | | |
| Aspirin | 500 mg to 1 g | | 1 | g | 500 mg |
| Acetaminophen | 1 | g | 1.3 | g | 300 mg |
| Ibuprofen | 400 | mg | 400 | mg | 150 to |
| | | | | | 200 mg, |
| | | | | | 150 mg, |
| | | | | | or 20 mg |
| Metamizole | 1 | g | 1 | g | |
| Caffeine | 130 | mg | | | 50 to |
| | | | | | 120 mg, |
| | | | | | 50 mg, |
| | | | | | 100 mg, |
| | | | | | or 120 mg |
| Chlorpheniramine | 4 to 12 | mg | | | 2.5 to |
| | | | | | 4 mg, |
| | | | | | 2.5 mg, |
| | | | | | or 4 mg |

| | | |
|---|---|---|
| Diphenhydramine | 25 to 50 mg | 25 to |
| hydrochloride | (hydrochloride) | 50 mg, |
| | | 25 mg, |
| | | 30 mg, |
| | | or 50 mg |
| Vitamin B1 and | | 10 mg |
| derivatives | | |
| thereof | | |

These pharmacologically active substances can generally cause not only problems relating to unpleasant taste and/or unpleasant odor but also problems relating to stability such as discoloration over time, but the present invention can provide a coated solid pharmaceutical preparation which not only makes it difficult to perceive the unpleasant taste and/or unpleasant odor of pharmacologically active substances when taken but also does not easily discolor over time, as described above.

Further, examples of the pharmacologically active substance which can cause problems relating to stability other than above include azulene, ethyl aminobenzoate, benzoic acids, ambroxol, camphor, salicylic acids, magnesium oxide, baking soda, magnesium hydroxide, tipepidine, Neusilin, vitamin B6, vitamin B12, vitamin D, vitamin E, menthol, and funnel extract.

Further, examples of the pharmacologically active substance other than above can also include pharmacologically active substances generally contained in a gastric-soluble preparation, an enteric-soluble preparation, or a sustained release preparation (which will be hereinafter referred to also as other pharmacologically active substances). The coated solid pharmaceutical preparation of the disclosure tends to allow gastric or enteric solubility, or sustained releasability to be exerted. In particular, the gastric or enteric solubility, or sustained releasability tends to be exerted even with a less amount of the coating film, and the production time, the energy consumption, and the amount of raw material used can be expected to be reduced.

Examples of the other pharmacologically active substances include isosorbide, isoprenaline, isopropamide, ibudilast, indomethacin, urapidil, esomeprazole, ethynyl estradiol, emedastine, potassium chloride, oxycodone, omeprazole, captopril, carteolol, quetiapine, chlormadinone, salazosulfapyridine, salicylamide, dioctyl sodium sulfosuccinate, diclofenac, disopyramide, dipyridamole, diprophylline, scopolamine, cephalexin, tacrolimus, tapentadol, tamsulosin, tolterodine, nicardipine, nifedipine, barnidipine, sodium valproate, paroxetine, bisacodyl, hydromorphone, pyridoxal, pyridoxine or salts thereof, pindolol, pheniramine, fesoterodine, bunazosin, ferrous fumarate, flavin adenine dinucleotide, pramipexole, furosemide, bezafibrate, belladonna total alkaloids, beraprost, vonoprazan, mesalazine, methylphenidate, metoprolol, morphine, soluble ferric pyrophosphate, rabeprazole, lansoprazole, iron sulfate, and roxatidine.

Among the pharmacologically active substances, ascorbic acid, aspirin, acetaminophen, ibuprofen, ethenzamide, epinastine, ephedrines, caffeine, Chinese herbal extracts, chlorpheniramine, chondroitin sulfate, Na chondroitin sulfate, cysteine, methionine, diphenhydramine, crude drug extracts, theophylline, tranexamic acid, noscapine, Ca pantothenate, vitamin B1 and derivatives thereof, vitamin C, pyridoxine or salts thereof, pirenzepine hydrochloride, fexofenadine, and meloxicam are preferable, and ascorbic acid, ibuprofen, cysteine, and pyridoxine or salts thereof are particularly preferable.

Only one of the pharmacologically active substances may be used alone, or two or more of them may be used in combination.

The content of the pharmacologically active substances in the coated solid pharmaceutical preparation of the disclosure is preferably 1 mass % or more and 99 mass % or less, more preferably 5 mass % or more and 95 mass % or less, even more preferably 20 mass % or more and 90 mass % or less, even more preferably 30 mass % or more and 85 mass % or less. In the case where the dosage form is a tablet or capsule, the content is even more preferably 40 mass % or more and 85 mass % or less, even more preferably 50 mass % or more and 80 mass % or less, particularly preferably 60 mass % or more and 80 mass % or less. Meanwhile, in the case where the dosage form is granules, the content is even more preferably 30 mass % or more and 70 mass % or less, even more preferably 30 mass % or more and 60 mass % or less, particularly preferably 30 mass % or more and 50 mass % or less.

The solid pharmaceutical preparation may contain a pharmaceutical additive, as required, in addition to the above pharmacologically active substances.

Examples of the pharmaceutical additive include one or more selected from the group consisting of stabilizers, surfactants (e.g., sodium lauryl sulfate, sucrose fatty acid ester, polyoxyethylene polyoxypropylene glycol, and polyoxyl stearate), plasticizers, lubricants (e.g., talc, magnesium stearate, calcium stearate, sodium stearyl fumarate, and glycerin fatty acid ester), solubilizers, buffers, sweeteners, bases, adsorbents, flavoring agents, binders (e.g., hydroxypropylmethylcellulose, hydroxypropylcellulose, gelatin, and alfarized starch), suspending agents, antioxidants, brighteners, fragrances, sustaining agents, humectants, humidity modifiers, defoamers, cooling agents, antistatic agents, colorants, flavors, fragrances, isotonizing agents, softeners, emulsifiers, viscous agents, foaming agents, excipients (e.g., lactose, milk sugar hydrate, white sugar, glucose, mannitol, sorbitol, xylitol, corn starch, and crystalline cellulose), pH modifiers, dispersants, disintegrants (e.g., croscarmellose sodium, carmellose, carmellose calcium, crospovidone, low-substituted hydroxypropylcellulose, and sodium carboxymethyl starch), disintegration aids, antiseptics, preservatives, solubilizers, solubilizing agents, solvents, and fluidizing agents (e.g., light anhydrous silicic acid and aqueous silicon dioxide). Examples of these pharmaceutical additives specifically include those described in Encyclopedia of Pharmaceutical Additives 2016 (edited by the International Pharmaceutical Excipients Council Japan, YAKUJI NIPPO LIMITED.) and the web site (http://www.jpec.gr.jp/) of the International Pharmaceutical Excipients Council Japan issued from the Director of Pharmaceutical and Food Safety Bureau, Ministry of Health, Labor and Welfare No. 1204-1 (The Pharmaceutical Affairs Law).

Further, the content mass ratio of pharmacologically active substances in the solid pharmaceutical preparation to pharmaceutical additives in the solid pharmaceutical preparation, [(pharmacologically active substances)/(pharmaceutical additives)], is preferably 0.01 or more, more preferably 0.05 or more, even more preferably 0.1 or more, particularly preferably 0.3 or more, in view of the effect of suppressing the unpleasant taste and/or unpleasant odor and the discoloration or the like. Further, it is preferably 20 or less, more preferably 16 or less, even more preferably 8 or less, particularly preferably 4 or less, in view of the effect of suppressing the unpleasant taste and/or unpleasant odor and the discoloration or the like. The specific range is preferably 0.01 or more and 20 or less, more preferably 0.05 or more and 16 or less, even more preferably 0.1 or more and 8 or less, particularly preferably 0.3 or more and 4 or less.

The content of the solid pharmaceutical preparation in the coated solid pharmaceutical preparation of the disclosure is preferably 70 mass % or more and 99.9 mass % or less, more preferably 80 mass % or more and 99.9 mass % or less, even more preferably 85 mass % or more and 99.9 mass % or less, even more preferably 90 mass % or more and 99.5 mass % or less, particularly preferably 95 mass % or more and 98 mass % or less, in view of the effect of suppressing the unpleasant taste and/or unpleasant odor and the discoloration or the like.

The film-forming polymer in the specific film is not specifically limited as long as it is a polymer used for forming a film on a solid pharmaceutical preparation by spray coating or the like, and examples thereof include one or more selected from the group consisting of water-soluble polymers, water-insoluble polymers, gastric-soluble polymers, and enteric-soluble polymers. Among these, water-soluble polymers and water-insoluble polymers are preferable, in view of the effect of suppressing the unpleasant taste and/or unpleasant odor and the discoloration or the like.

Examples of the water-soluble polymers include cellulose-based water-soluble polymers, polyalkylene glycol-based water-soluble polymers, polyvinyl alcohol-based water-soluble polymers, and polyvinylpyrrolidone-based water-soluble polymers. Among these, cellulose-based water-soluble polymers and polyvinyl alcohol-based water-soluble polymers are preferable, and cellulose-based water-soluble polymers and polyvinyl alcohol-(meth)acrylic acid-(meth)alkyl acrylate copolymer-based water-soluble polymers are more preferable, in view of the effect of suppressing the unpleasant taste and/or unpleasant odor and the discoloration or the like.

Examples of the cellulose-based water-soluble polymers include methylcelluloses (e.g., Methocel Premium A and METOLOSE), hypromelloses (alias: hydroxypropylmethylcelluloses (e.g., Methocel Premium K, Methocel Premium F, Methocel Premium E, METOLOSE SR, TC-5, and Benecel)), and hydroxypropylcelluloses (e.g., Kulcel).

Examples of the polyalkylene glycol-based water-soluble polymers include macrogol (alias: polyethylene glycol).

Examples of the polyvinyl alcohol-based water-soluble polymers include polyvinyl alcohol-acrylic acid-methyl methacrylate copolymers (e.g., POVACOAT), polyvinyl alcohol partially saponified products (e.g., GOHSENOL, Kuraray Poval, and J POVAL), polyvinyl alcohol completely saponified products (alias: POVAL), and polyvinyl alcohol-polyethylene glycol-graft copolymers (e.g., Kollicoat IR). Examples of the polyvinylpyrrolidone-based water-soluble polymers include povidone (alias: polyvidone or polyvinylpyrrolidone (e.g., polyvinylpyrrolidone K25, polyvinylpyrrolidone K30, and polyvinylpyrrolidone K90)), and copolyvidone (e.g., Kollidon VA64 and Plasdone S-630).

Examples of the water-insoluble polymers include alkyl (meth)acrylate-based water-insoluble polymers and cellulose-based water-insoluble polymers. Among these, alkyl (meth)acrylate-based water-insoluble polymers are preferable, and ammonium salt-type cationic functional group-containing alkyl (meth)acrylate-based water-insoluble polymers are more preferable, in view of the effect of suppressing the unpleasant taste and/or unpleasant odor and the discoloration or the like.

Examples of the alkyl (meth)acrylate-based water-insoluble polymers include ethyl acrylate-methyl methacrylate copolymers (e.g., Eudragit NE30D), ethyl acrylate-methyl methacrylate-trimethylammonium ethyl methacrylate chloride copolymers (alias: aminoalkyl methacrylate copolymers RS (e.g., Eudragit RS100, Eudragit RSPO, Eudragit RL, Eudragit RLPO, Eudragit RS30D, and Eudragit RL30D)).

Examples of the cellulose-based water-insoluble polymers include ethylcelluloses (e.g., ETHOCEL, Aquacoat, SELiOS coat, and Surerease).

Examples of the gastric-soluble polymers include polyvinyl acetal-based gastric-soluble polymers such as polyvinyl acetal diethylaminoacetates (e.g., AEA); and (meth)acrylic acid-based gastric-soluble polymers such as methyl methacrylate-butyl methacrylate-dimethylaminoethyl methacrylate copolymers (alias: aminoalkyl methacrylate copolymers E (e.g., Eudragit EPO and Eudragit E100)) and methyl methacrylate-diethylaminoethyl methacrylate copolymers (e.g., Kollicoat Smartseal 30D).

Examples of the enteric-soluble polymers include cellacephates (alias: cellulose acetate phthalates (e.g., CAP and Aquateric)), hypromellose phthalic acid esters (alias: hydroxypropyl methylcellulose phthalates (e.g., HP-55 and HP-50)), hypromellose acetic acid ester succinic acid esters (alias: hydroxypropyl methylcellulose acetate succinates (e.g., AQOAT)), and carboxymethyl ethylcelluloses (e.g., CMEC), in addition to (meth)acrylic acid-based enteric-soluble polymers such as methacrylic acid-ethyl acrylate copolymers (alias: methacrylic acid copolymers LD (e.g., Eudragit L30D-55 and Eudragit L100-55)), methacrylic acid-methyl methacrylate copolymers (alias: methacrylic acid copolymers L (e.g., Eudragit L100), alias: methacrylic acid copolymers S (e.g., Eudragit S100)), and methyl acrylate-methyl methacrylate-methacrylic acid copolymers (e.g., Eudragit FS30D).

Only one of the film-forming polymers may be used alone, or two or more of them may be used in combination.

The content of the film-forming polymers in the coated solid pharmaceutical preparation of the disclosure is preferably 0.01 mass % or more and 15 mass % or less, more preferably 0.05 mass % or more and 12.5 mass % or less, even more preferably 0.1 mass % or more and 10 mass % or less, even more preferably 0.3 mass % or more and 7.5 mass % or less, even more preferably 0.5 mass % or more and 5 mass % or less, particularly preferably 1 mass % or more and 3 mass % or less, in view of the effect of suppressing the unpleasant taste and/or unpleasant odor and the discoloration or the like.

The content of the film-forming polymers in the specific film is preferably 30 mass % or more and 99.9 mass % or less, more preferably 50 mass % or more and 99.9 mass % or less, even more preferably 60 mass % or more and 99.9 mass % or less, particularly preferably 70 mass % or more and 99.9 mass % or less, in view of the effect of suppressing the unpleasant taste and/or unpleasant odor and the discoloration or the like.

Further, the content mass ratio of film-forming polymers in the specific film to pharmacologically active substances in the solid pharmaceutical preparation, [(film-forming polymers)/(pharmacologically active substances)], is preferably 0.001 or more, more preferably 0.003 or more, even more preferably 0.01 or more, particularly preferably 0.015 or more, in view of the effect of suppressing the unpleasant taste and/or unpleasant odor and the discoloration or the like. Further, it is preferably 0.2 or less, more preferably 0.1 or less, even more preferably 0.075 or less, particularly preferably 0.05 or less, in view of the effect of suppressing the unpleasant taste and/or unpleasant odor and the discoloration or the like. The specific range is preferably 0.001 or more and 0.2 or less, more preferably 0.003 or more and 0.1 or less, even more preferably 0.01 or more and 0.075 or less, particularly preferably 0.015 or more and 0.05 or less.

The phrase “flaky substance” as used herein means a scaly strip-shaped substance with a particle size larger than the thickness.

The flaky substance is preferably a flaky substance containing a silicon atom. Examples thereof include potassium aluminum silicates (mica and sericite), fine silicon dioxides (flaky silica), aluminum silicates (colloidal hydrous aluminum silicate (bentonite) and hydrous aluminum silicate (kaolin)), and magnesium aluminum silicates. The flaky substances coated with a coloring pigment such as iron oxide or titanium oxide also can be used. The coating amount of the coloring pigment is preferably 10 to 150 parts by mass with respect to 100 parts by mass of the flaky substance. Only one of the flaky substances may be used alone, or two or more of them may be used in combination.

Among these flaky substances, potassium aluminum silicates and fine silicon dioxides are preferable, in view of the effect of suppressing the unpleasant taste and/or unpleasant odor and the discoloration or the like.

The flaky substances have an average particle size of generally in the range from 0.2 to 500 μm, preferably in the range from 0.5 to 300 μm, more preferably in the range from 1 to 150 μm.

Further, the flaky substances have a thickness of generally in the range from 10 to 5000 nm, preferably in the range from 50 to 2000 nm, more preferably in the range from 100 to 1200 nm.

Further, the flaky substances have an aspect ratio of generally in the range from 5 to 500, preferably in the range from 10 to 300, more preferably in the range from 15 to 150.

The average particle size of the flaky substances is a sphere-equivalent average particle size measured by the laser diffraction scattering method, and the average particle size, the thickness, and the aspect ratio can be measured by the dynamic image analysis method.

The content of the flaky substances in the coated solid pharmaceutical preparation of the disclosure is preferably 0.0001 mass % or more and 20 mass % or less, more preferably 0.0005 mass % or more and 10 mass % or less, even more preferably 0.001 mass % or more and 5 mass % or less, even more preferably 0.01 mass % or more and 3 mass % or less, particularly preferably 0.1 mass % or more and 1 mass % or less, in view of the effect of suppressing the unpleasant taste and/or unpleasant odor and the discoloration or the like.

The content of the flaky substances in the specific film is preferably 0.001 mass % or more and 70 mass % or less, more preferably 0.01 mass % or more and 50 mass % or less, even more preferably 0.1 mass % or more and 40 mass % or less, particularly preferably 10 mass % or more and 30 mass % or less, in view of the effect of suppressing the unpleasant taste and/or unpleasant odor and the discoloration or the like.

Further, the content mass ratio of flaky substances in the specific film to pharmacologically active substances in the solid pharmaceutical preparation, [(flaky substances)/(pharmacologically active substances)], is preferably 0.00001 or more, more preferably 0.0001 or more, even more preferably 0.001 or more, particularly preferably 0.005 or more, in view of the effect of suppressing the unpleasant taste and/or unpleasant odor and the discoloration or the like. Further, it is preferably 1 or less, even more preferably 0.5 or less, even more preferably 0.1 or less, particularly preferably 0.05 or less, in view of the effect of suppressing the unpleasant taste and/or unpleasant odor and the discoloration or the like. The specific range is preferably 0.00001 or more and 1 or less, more preferably 0.0001 or more and 0.5 or less, even more preferably 0.001 or more and 0.1 or less, particularly preferably 0.005 or more and 0.05 or less.

Further, the content mass ratio of flaky substances in the specific film to film-forming polymers in the specific film, [(flaky substances)/(film-forming polymers)], is preferably 0.0002 or more, more preferably 0.002 or more, even more preferably 0.02 or more, particularly preferably 0.1 or more, in view of the effect of suppressing the unpleasant taste and/or unpleasant odor and the discoloration or the like. Further, it is preferably 20 or less, more preferably 10 or less, even more preferably 5 or less, particularly preferably 1 or less, in view of the effect of suppressing the unpleasant taste and/or unpleasant odor and the discoloration or the like. The specific range is preferably 0.0002 or more and 20 or less, more preferably 0.002 or more and 10 or less, even more preferably 0.02 or more and 5 or less, particularly preferably 0.1 or more and 1 or less.

The specific film may contain a pharmaceutical additive, as required, other than film-forming polymers and flaky substances.

Examples of the pharmaceutical additive include plasticizers, coating agents, dispersants, colorants, and defoamers. Examples of these pharmaceutical additives specifically include those described in Encyclopedia of Pharmaceutical Additives 2016 (edited by the International Pharmaceutical Excipients Council Japan, YAKUJI NIPPO LIMITED.) and the web site (http://www.jpec.gr.jp/) of the International Pharmaceutical Excipients Council Japan issued from the Director of Pharmaceutical and Food Safety Bureau, Ministry of Health, Labor and Welfare No. 1204-1 (The Pharmaceutical Affairs Law). Only one of the pharmaceutical products additives may be used alone, or two or more of them may be used in combination.

Specific examples of the plasticizers include carillon 83, triethyl citrate, glycerin, glycerin fatty acid ester, sesame oil, dimethyl polysiloxane-silicon dioxide mixture, D-sorbitol, medium-chain fatty acid triglyceride, corn starch-derived sugar alcohol liquid, triacetin, concentrated glycerin, castor oil, diethyl phthalate, dibutyl phthalate, butylphthalylbutylglycolate, polyoxyethylene (105) polyoxypropylene (5) glycol, propylene glycol, polysorbate 80, macrogol 400, macrogol 600, macrogol 1500, macrogol 4000, macrogol 6000, cottonseed oil-soybean oil mixture, and glycerin monostearate. Only one of these may be used alone, or two or more of them may be used in combination.

The content of the plasticizers in the specific film is generally 30 mass % or less, preferably 25 mass % or less.

Specific examples of the coating agents include olive oil, cacao butter, prunellae spica, castor wax, caramel, carnaubaro, carboxyvinyl polymer, carboxymethyl ethylcellulose, sodium carboxymethyl starch, carmellose calcium, carmellose sodium, dry aluminum hydroxide gel, dry milky white lac, Kanbaiko, fish scale, gold foil, silver foil, triethyl citrate, glycerin, glycerin fatty acid ester, magnesium silicate, light anhydrous silicic acid, hydroxypropylcellulose containing light anhydrous silicic acid, light liquid paraffin, spermaceti, crystalline cellulose, hardened oil, synthetic wax, high glucose water candy, hard wax, amber gelatin, flour, wheat starch, rice starch, white beeswax, titanium oxide, magnesium oxide, dimethyl polysiloxane (for oral administration), dimethyl polysiloxane-silicon dioxide mixture, grilled plaster, sucrose fatty acid ester, powdered eagle wood, aluminum hydroxide gel, hydrogenated rosin glycerol ester, stearyl alcohol, stearic acid, aluminum stearate, calcium stearate, polyoxyl stearate 40, magnesium stearate, purified gelatin, purified shellac, purified white sugar, zein, sorbitan sesquioleate, cetanol, plaster, gelatin, shellac, sorbitan fatty acid ester, D-sorbitol, D-sorbitol liquid, tricalcium phosphate, talc, calcium carbonate, magnesium carbonate, simple syrup, medium gold foil, precipitated calcium carbonate, low-substituted hydroxypropylcellulose, terpene resin, starch (soluble), corn syrup, corn oil, triacetin, calcium lactate, lactose, concentrated glycerin, white shellac, white sugar, honey, paraffin, pearl powder, potato starch, castor oil, diethyl phthalate, dibutyl phthalate, butylphthalylbutylglycolate, glucose, pullulan, propylene glycol, povidone, polyoxyethylene hardened castor oil 40, polyoxyethylene hardened castor oil 60, polyoxyethylene (105) polyoxypropylene (5) glycol, polyoxyethylene (160) polyoxypropylene (30) glycol, polysorbate 80, macrogol 300, macrogol 400, macrogol 600, macrogol 1500, macrogol 1540, macrogol 4000, macrogol 6000, macrogol 20000, macrogol 35000, D-mannitol, water candy, beeswax, myristyl alcohol, phthalic anhydride, anhydrous calcium hydrogen phosphate, Japan wax, aluminum monostearate, glycerin monostearate, sorbitan monolaurate, montanic acid ester wax, medicinal charcoal, lauromacrogol, calcium sulfate, liquid paraffin, DL-malic acid, calcium monohydrogen phosphate, calcium hydrogen phosphate, sodium hydrogen phosphate, calcium dihydrogen phosphate, and rosin. Only one of these may be used alone, or two or more of them may be used in combination.

The content of the coating agents in the specific film is generally 30 mass % or less, preferably 20 mass % or less.

Specific examples of the dispersants include gum arabic, gum arabic powder, propylene glycol alginate ester, ethanol, oleic acid, carboxyvinyl polymer, carmellose sodium, agar powder, citric acid, sodium citrate, glycerin, glycerin fatty acid ester, magnesium silicate, light anhydrous silicic acid, crystalline cellulose, hardened oil, choline phosphate, safflower oil, white beeswax, titanium oxide, dioctyl sodium sulfosuccinate, sucrose fatty acid ester, sodium hydroxide, stearic acid, magnesium stearate, purified oleic acid, purified soy lecithin, sorbitan sesquioleate, sorbitan fatty acid ester, D-sorbitol, soybean oil, soybean lecithin, low-substituted hydroxypropylcellulose, dextrin, corn starch, tragacanth powder, sorbitan trioleinate, lactose, concentrated glycerin, potato starch, propylene glycol, propylene glycol fatty acid ester, povidone, polyoxyethylene hardened castor oil, polyoxyethylene hardened castor oil 40, polyoxyethylene hardened castor oil 60, polyoxyethylene (105) polyoxypropylene (5) glycol, polyoxyethylene (160) polyoxypropylene (30) glycol, polysorbate 20, polysorbate 60, polysorbate 80, sodium polyphosphate, macrogol 300, macrogol 4000, macrogol 6000, anhydrous sodium citrate, anhydrous sodium pyrophosphate, magnesium aluminometasilicate, sodium metaphosphate, methylcellulose, Japan wax, sorbitan monooleate, aluminum monostearate, glycerin monostearate, sorbitan monopalmitate, sorbitan monolaurate, sodium lauryl sulfate, lauromacrogol, liquid paraffin, and calcium hydrogen phosphate. Only one of these may be used alone, or two or more of them may be used in combination.

The content of the dispersants in the specific film is generally 25 mass % or less, preferably 15 mass % or less.

Specific examples of the colorants include Asenyaku tannin (catechtannic acid) powder, turmeric extract, yellow iron sesquioxide, OPA spray K-1-24904, orange essence, brown iron oxide, carbon black, caramel, carmine, carotene liquid, β-carotene, licorice extract, gold foil, black iron oxide, light anhydrous silicic acid, titanium oxide, iron sesquioxide, edible blue No. 1, edible yellow No. 4, edible yellow No. 4 aluminum lake, edible yellow No. 5, edible red No. 2, edible red No. 3, edible red No. 102, sodium hydroxide, talc, copper chloro fin sodium, copper chlorophyll, hadakamugi green leaves extract, d-borneol, octyldodecyl myristate, medicinal charcoal, riboflavin butyrate, riboflavin, green tea powder, riboflavin sodium phosphate, and rose oil. Only one of these may be used alone, or two or more of them may be used in combination.

The content of the colorants in the specific film is generally 10 mass % or less, preferably 5 mass % or less.

Specific examples of the defoamers include ethanol, glycerin fatty acid ester, dimethylpolysiloxane (for oral administration), dimethylpolysiloxane-silicon dioxide mixture, sucrose fatty acid ester, silicone resin emulsion, silicone defoamers, polyoxyl stearate 40, sorbitan fatty acid ester, sorbitan trioleinate, and polysorbate 80. Only one of these may be used alone, or two or more of them may be used in combination.

The content of the defoamers in the specific film is generally 5 mass % or less, preferably 1 mass % or less.

The thickness and the coating amount of the specific film may be adjusted depending on the type of film-forming polymer or the type and the dosage form of the solid pharmaceutical preparation, but the thickness of the specific film is generally 0.1 μm or more, preferably 1 μm or more, particularly preferably 2 μm or more. For example, in the case of a tablet with a diameter of about 9 mm and a mass of about 200 mg, the mass of 5 μm-thick film is about 1 to 2 mg/tablet.

Further, the mass ratio of the coating amount of the specific film to the solid pharmaceutical preparation, [(specific film)/(solid pharmaceutical preparation)], is preferably 0.0001 or more, more preferably 0.001 or more, even more preferably 0.005 or more, particularly preferably 0.01 or more, in view of the effect of suppressing the unpleasant taste and/or unpleasant odor and the discoloration or the like. Further, it is preferably 1 or less, more preferably 0.75 or less, even more preferably 0.5 or less, particularly preferably 0.1 or less, in view of the effect of suppressing the unpleasant taste and/or unpleasant odor and the discoloration or the like. The specific range is preferably 0.0001 or more and 1 or less, more preferably 0.001 or more and 0.75 or less, even more preferably 0.005 or more and 0.5 or less, particularly preferably 0.01 or more and 0.1 or less. The coated solid pharmaceutical preparation of the disclosure makes it difficult to perceive the unpleasant taste and/or unpleasant odor of pharmacologically active substances when taken and does not easily discolor over time, even with a less amount of coating, as above.

The total content of the solid pharmaceutical preparation and the specific film is preferably 75 mass % or more and 100 mass % or less, more preferably 80 mass % or more and 100 mass % or less, even more preferably 85 mass % or more and 100 mass % or less, particularly preferably 90 mass % or more and 100 mass % or less, in view of the effect of suppressing the unpleasant taste and/or unpleasant odor and the discoloration or the like in the coated solid pharmaceutical preparation of the disclosure.

The coated solid pharmaceutical preparation of the disclosure may include a film other than the specific film (this film will be hereinafter referred to as “other film”). The other film is the same as the specific film except that it is free from flaky substances. Further, the other film may be applied between the solid pharmaceutical preparation and the specific film or may be applied onto the outer side of the specific film.

The phrase “coated solid pharmaceutical preparation” as used herein refer to a pharmaceutical preparation in which a film is applied to a solid pharmaceutical preparation. The film may be applied to the solid pharmaceutical preparation directly or via another film.

The coated solid pharmaceutical preparation is preferably for oral use.

Examples of the dosage forms of the solid pharmaceutical preparation and the coated solid pharmaceutical preparation include solid forms described in the general rules of the 17th edition of the Japanese Pharmacopoeia for preparations orally administered and preparations orally applied. Specifically, example thereof include tablets, capsules, granules, powders, solid syrups, orally jellies, and oral tablets. Tablets include orally disintegrating tablet, chewable tablets, effervescent tablets, dispersible tablets, soluble tablets, in addition to general tablets. Further, oral tablets include lozenges, sublingual tablets, buccal tablets, adhesive tablets, and gum agents. Further, capsules include soft capsules other than hard capsules. Further, granules include fine granules and effervescent granules.

Among these dosage forms, tablets, capsules, granules, and powders are preferable.

The coated solid pharmaceutical preparation of the disclosure can be produced by appropriately combining conventional methods. A solid pharmaceutical preparation comprising a pharmacologically active substance may be formulated by a general method and coated with a specific film. Further, it may be coated with the other film before and/or after coating with the specific film, as required.

In the case where granule powder needs to be prepared in the production of tablets, capsules, granules, or powders as a solid pharmaceutical preparation, the granule powder can be produced by a generally used granulation method (e.g., wet granulation methods such as the spray granulation method using a solution or dispersion containing water or an organic solvent, the stirring granulation method, the fluidized granulation method, the rolling granulation method, and the rolling fluidized granulation method, and dry granulation methods such as the consolidation granulation method using powdery granular binders). Tablets can be produced by mixing the granule powder and a pharmaceutical additive, as required, and compression-forming the mixture. Capsules can be produced by mixing the granule powder or tablet and a pharmaceutical additive, as required, and filling a hard capsule or soft capsule with the mixture.

Further, the specific film can be applied by coating the solid pharmaceutical preparation obtained, as described above, with a film-forming polymer, a flaky substance, and a pharmaceutical additive, as required. The coating method is not specifically limited, and examples thereof include the pan-coating method, the fluidized bed coating method, the rolling coating method, the dry coating method, and combinations thereof. More specifically, examples thereof include a coating method using a solution or suspension obtained by adding a composition containing a film-forming polymer, a flaky substance, and a pharmaceutical additive, as required, to a solvent such as purified water or ethanol.

The coated solid pharmaceutical preparation of the disclosure not only makes it difficult to perceive the unpleasant taste and/or unpleasant odor of pharmacologically active substances when taken but also does not easily discolor over time. Further, the coated solid pharmaceutical preparation of the disclosure is excellent in the dissolution properties or disintegration characteristics.

Further, the sealability (shielding property) is improved without impairing the functionality of the film-forming polymer such as water solubility, gastric solubility, enteric solubility, and sustained release. Therefore, it can be expected to suppress the occurrence of unpleasant odor over time, prevent sublimation, improve stability, and suppress changes in the composition. Further, the same effect is exerted, even with a less amount of the film to be coated. Accordingly, the production time is shortened, and further the energy consumption and CO2 emission, the amount of raw material to be used also are reduced, thereby enabling production at a low cost with a low environmental load.

<Method for Suppressing Unpleasant Taste and/or Unpleasant Odor and Method for Suppressing Discoloration>

The method for suppressing unpleasant taste and/or unpleasant odor derived from a pharmacologically active substance when a solid pharmaceutical preparation comprising the pharmacologically active substance is taken of the disclosure comprises applying a film comprising a film-forming polymer and a flaky substance to the solid pharmaceutical preparation.

The method for suppressing discoloration over time when a solid pharmaceutical preparation comprising a pharmacologically active substance is stored of the disclosure comprises applying a film comprising a film-forming polymer and a flaky substance to the solid pharmaceutical preparation.

The meaning of various wordings, the content of each component, and the ratio thereof, etc., in the method for suppressing unpleasant taste and/or unpleasant odor and the method for suppressing discoloration of the present invention are the same as those described for the coated solid pharmaceutical preparation of the disclosure.

### EXAMPLES

Hereinafter, the present invention will be described in detail by way of Examples. However, the present invention is not limited to the following Examples.

### Example 1-1 to Example 1-4

(1) Production of Solid Pharmaceutical Preparation (Uncoated Tablet)

Tableting powder was produced using 1,920 g of L-cysteine (available from NIPPON RIKA CO., LTD.), 4000 g of ascorbic acid (available from FUSO CHEMICAL CO., LTD), 400 g of pyridoxine hydrochloride (available from BASF SE), 1580 g of crystalline cellulose (CEOLUS (available from Asahi Kasei Corporation)), 320 g of low-substituted hydroxypropylcellulose (L-HPC, available from Shin-Etsu Chemical Co., Ltd.), 24 g of light anhydrous silicic acid (available from Freund Corporation), and 112 g of calcium stearate (available from Merck KGaA) by a conventional method. The mixed powder obtained was tableted to a mass of 265 mg and a thickness of 4.7 mm per tablet by a rotary tableting machine (VIRGO-0512 tableting machine, available from KIKUSUI SEISAKUSHO LTD.) with an 8.5-mm-diameter mortar and pestle, to obtain about 8.2 kg of an uncoated tablet.

(2) Production of Coated Solid Pharmaceutical Preparation (Film-Coated Tablet) of the Disclosure

60 g of a film-forming polymer (polyvinyl alcohol-acrylic acid-methyl methacrylate copolymer: POVACOAT, available from Nisshin Kasei Co., Ltd.) and 20 g of a flaky substance (titanium oxide (41.2%)-coated potassium aluminum silicate (58.8%) with an average particle size of 6.2 μm (laser diffraction scattering method), available from Merck KGaA) were dissolved and suspended in 1,500 g of purified water to prepare a film-coating solution. 350 g of the uncoated tablet obtained in (1) above was coated with the film-coating solution using a coating machine (HICOATER HC-LABO, available from Freund Corporation) to a mass gain per uncoated tablet of 1 mg (Example 1-1), 2 mg (Example 1-2), 5 mg (Example 1-3), or 10 mg (Example 1-4) to obtain the coated solid pharmaceutical preparation of the disclosure as a film-coated tablet.

### Comparative Example 1-1 to Comparative Example 1-4

60 g of a film-forming polymer (polyvinyl alcohol-acrylic acid-methyl methacrylate copolymer: POVACOAT, available from Nisshin Kasei Co., Ltd.), 20 g of titanium oxide (available form ISHIHARA SANGYO KAISHA, LTD.) were dissolved and suspended in 1,500 g of purified water to prepare a film-coating solution. 350 g of uncoated tablet obtained in the same manner as in Example 1 (1) was coated with the film-coating solution using a coating machine (HICOATER HC-LABO, available from Freund Corporation) to a mass gain per uncoated tablet of 1 mg (Comparative Example 1-1), 2 mg (Comparative Example 1-2), 5 mg (Comparative Example 1-3), or 10 mg (Comparative Example 1-4) to obtain each comparative coated solid pharmaceutical preparation as a film-coated tablet.

### Test Example 1: Test for Deodorant Effect

The coated solid pharmaceutical preparations produced in Example 1-1 to Example 1-4 and Comparative Example 1-1 to Comparative Example 1-4 and 30 uncoated tablets obtained in Example 1 (1) were each put into a glass No. 5 standard bottle, and a stopper was thereon to seal. After storage at 25° C. for 24 hours, the bottle was opened, and the cysteine odor at that time was evaluated according to the following evaluation criteria by 12 test subjects to determine an average score. Table 2 shows the results.

<Evaluation Criteria for Effect of Suppressing Unpleasant Odor>
0 points: No cysteine odor
1 point: Cysteine odor slightly perceived
2 points: Cysteine odor perceived
3 points: Cysteine odor strongly perceived

TABLE 2

| | | | | | Compar- | Compar- | Compar- | Compar- | |
|---|---|---|---|---|---|---|---|---|---|
| | | | | | ative | ative | ative | ative | Un- |
| | Example | Example | Example | Example | Example | Example | Example | Example | coated |
| | 1-1 | 1-2 | 1-3 | 1-4 | 1-1 | 1-2 | 1-3 | 1-4 | tablet |
| Coating amount | 1 | 2 | 5 | 10 | 1 | 2 | 5 | 10 | 0 |
| | mg/tablet | mg/tablet | mg/tablet | mg/tablet | mg/tablet | mg/tablet | mg/tablet | mg/tablet | mg/tablet |
| Film-forming | 0.75 | 1.5 | 3.75 | 7.5 | 0.75 | 1.5 | 3.75 | 7.5 | — |
| polymer | mg/tablet | mg/tablet | mg/tablet | mg/tablet | mg/tablet | mg/tablet | mg/tablet | mg/tablet | |
| Flaky substance | 0.25 | 0.5 | 1.25 | 2.5 | — | — | — | — | — |
| | mg/tablet | mg/tablet | mg/tablet | mg/tablet | | | | | |
| Test subject 1 | 1 | 0 | 0 | 0 | 2 | 2 | 1 | 1 | 3 |
| Test subject 2 | 1 | 0 | 0 | 0 | 3 | 2 | 1 | 0 | 3 |
| Test subject 3 | 1 | 0 | 0 | 0 | 3 | 2 | 2 | 1 | 3 |
| Test subject 4 | 1 | 0 | 0 | 0 | 3 | 2 | 2 | 1 | 3 |
| Test subject 5 | 1 | 0 | 0 | 0 | 2 | 2 | 1 | 1 | 3 |
| Test subject 6 | 0 | 0 | 0 | 0 | 2 | 2 | 1 | 1 | 3 |
| Test subject 7 | 1 | 0 | 0 | 0 | 3 | 2 | 1 | 0 | 3 |
| Test subject 8 | 1 | 0 | 0 | 0 | 2 | 2 | 1 | 1 | 3 |
| Test subject 9 | 1 | 0 | 0 | 0 | 3 | 2 | 2 | 1 | 3 |
| Test subject 10 | 1 | 0 | 0 | 0 | 3 | 2 | 2 | 1 | 3 |
| Test subject 11 | 1 | 0 | 0 | 0 | 3 | 2 | 2 | 1 | 3 |
| Test subject 12 | 0 | 0 | 0 | 0 | 2 | 2 | 1 | 1 | 3 |
| Average | 0.8 | 0.0 | 0.0 | 0.0 | 2.6 | 2.0 | 1.4 | 0.8 | 3.0 |
| Standard | 0.4 | 0.0 | 0.0 | 0.0 | 0.5 | 0.0 | 0.5 | 0.4 | 0.0 |
| deviation | | | | | | | | | |

As shown in Table 2, the coated solid pharmaceutical preparations (Example 1-1 to Example 1-4) with a film containing a flaky substance in addition to the film-forming polymer applied made it difficult to perceive unpleasant odor derived from the pharmacologically active substance.

Further, the coated solid pharmaceutical preparations of the disclosure made it difficult to perceive unpleasant odor, even with a very small coating amount of 1 mg/tablet. Further, for the coated solid pharmaceutical preparations of the disclosure, unpleasant odor was suppressed so that odor was not perceived with a coating amount of 2 mg or more per tablet.

### Test Example 2: Test for Effect of Preventing Discoloration

30 tablets of each of the coated solid pharmaceutical preparation of Example 1-4 and the coated solid pharmaceutical preparation of Comparative Example 1-4 were put into a glass No. 5 standard bottle, and a stopper was put thereon to seal. It was stored in a thermostatic chamber at 60° C. for 22 days, and the change in color tones at that time from before (Initial) the storage was measured with a spectrocolorimeter (SE 7700: available from NIPPON DENSHOKU INDUSTRIES CO., LTD.), to calculate ΔE (from the Initial). Table 3 shows the results.

TABLE 3

| | | Comparative |
|---|---|---|
| | Example 1-4 | Example 1-4 |
| Coating amount | 10 mg/tablet | 10 mg/tablet |
| Film-forming polymer | 7.5 mg/tablet | 7.5 mg/tablet |
| Flaky substance | 2.5 mg/tablet | — |
| ΔE (from the Initial) | 9.98 | 12.42 |

As shown in Table 3, it turned out that the coated solid pharmaceutical preparation (Example 1-4) in which a film containing a flaky substance in addition to the film-forming polymer was applied to a solid pharmaceutical preparation containing a pharmacologically active substance made it difficult to discolor over time and had excellent stability.

### Example 2

(1) Production of Tablet with Undercoating

48 g of a polyvinyl alcohol-acrylic acid-methyl methacrylate copolymer (POVACOAT, available from Nisshin Kasei Co., Ltd.), 10 g of titanium oxide (available form ISHIHARA SANGYO KAISHA, LTD.), and 22 g of talc (available from NIPPON TALC Co., Ltd.) were dissolved and suspended in 320 g of purified water to prepare a film-coating solution containing talc. 400 g of the uncoated tablet produced in Example 1 (1) was coated with the film-coating solution containing talc using a coating machine (HICOATER HC-LABO, available from Freund Corporation) to a mass gain per uncoated tablet of 20 mg to obtain a tablet with undercoating applied.
(2) Production of Coated Solid Pharmaceutical Preparation (Film-Coated Tablet) of the Disclosure

39 g of a film-forming polymer (hypromellose: TC-5, available from Shin-Etsu Chemical Co., Ltd.) and 1 g of a flaky substance (titanium oxide (25.3%)-coated fine silicon dioxide (74.7%) with an average particle size of 19.1 μm (laser diffraction scattering method), available from Merck KGaA) were dissolved and suspended in 1,500 g of purified water to prepare a film-coating solution containing a flaky substance.

350 g of the tablet with an undercoating applied was coated with the film-coating solution containing a flaky substance using a coating machine (HICOATER HC-LABO, available from Freund Corporation) to a mass gain per uncoated tablet of 1 mg to obtain the coated solid pharmaceutical preparation of the disclosure as a film-coated tablet. 1 mg/tablet of the film as the outermost layer of each preparation contains 0.975 mg/tablet of a film-forming polymer and 0.025 mg/tablet of a flaky substance.

As a result of a test for the deodorant effect in the same manner as in Test Example 1, the coated solid pharmaceutical preparation of Example 2 had an average score of the deodorant effect of 0.08, which was very excellent.

### Example 3-1 to Example 3-4

39.9 g of a film-forming polymer (hypromellose: TC-5, available from Shin-Etsu Chemical Co., Ltd.) and 0.1 g of a flaky substance (titanium oxide (41.2%)-coated potassium aluminum silicate (58.8%) with an average particle size of 6.2 μm (laser diffraction scattering method), available from Merck KGaA) were dissolved and suspended in 1,500 g of purified water to prepare a film-coating solution containing a flaky substance.

350 g of the tablet with an undercoating applied produced in Example 2 (1) was coated with the film-coating solution containing a flaky substance using a coating machine (HICOATER HC-LABO, available from Freund Corporation) to a mass gain per uncoated tablet of 1 mg (Example 3-1), 2 mg (Example 3-2), 3 mg (Example 3-3), or 4 mg (Example 3-4) to obtain the coated solid pharmaceutical preparation of the disclosure as a film-coated tablet.

The coated solid pharmaceutical preparations of Example 3-1 to Example 3-4 and the tablet with an undercoating applied produced in Example 2 (1) were subjected to a test for the deodorant effect in the same manner as in Test Example 1. Table 4 shows the results.

TABLE 4

| | | | | | | | | | Tablet with |
|---|---|---|---|---|---|---|---|---|---|
| | | | | | | | | | undercoating |
| | Example 3-1 | | Example 3-2 | | Example 3-3 | | Example 3-4 | | applied |
| Coating amount in | 1 | mg/tablet | 2 | mg/tablet | 3 | mg/tablet | 4 | mg/tablet | — |
| outermost layer | | | | | | | | | |
| Film-forming | 0.9975 | mg/tablet | 1.995 | mg/tablet | 2.9925 | mg/tablet | 3.99 | mg/tablet | — |
| polymer in | | | | | | | | | |
| outermost layer | | | | | | | | | |
| Flaky substance in | 0.0025 | mg/tablet | 0.005 | mg/tablet | 0.0075 | mg/tablet | 0.01 | mg/tablet | — |
| outermost layer | | | | | | | | | |

| | | | | | |
|---|---|---|---|---|---|
| Average score | 0.92 | 0.83 | 0.75 | 0.58 | 1.08 |

In the cases of combining hypromellose as a film-forming polymer and potassium aluminum silicate as a flaky substance (Example 3-1 to Example 3-4), the unpleasant odor derived from the pharmacologically active substance could be suppressed.

### Example 4

(1) Production of Granule

A kneading solution obtained by dissolving 40 g of polyoxyl stearate 40 (available from NOF CORPORATION) in 500 g of purified water was added to a mixture of 1,800 g of ibuprofen (available from BASF SE), 2,240 g of purified white sugar (available from Toyo Sugar Refining Co., Ltd.), 288 g of crystalline cellulose (available from Asahi Kasei Corporation), 320 g of corn starch (available from Nihon Cornstarch corporation), and 112 g of hydroxypropylcellulose (available from Nippon Soda Co., Ltd.). This composition was extruded and granulated by a conventional method, followed by drying, and the particle size was adjusted using 30-mesh and 42-mesh sieves to obtain a granule.

(2) Production of Granule (Coating Granule)

26.3 parts by mass of a film-forming polymer dispersion (ammonioalkyl methacrylate copolymer dispersion with a solid content of 30 mass %, available from Evonik Industries AG), 1.0 part by mass of a flaky substance (titanium oxide (12.3%)-coated potassium aluminum silicate (87.7%) with an average particle size of 77.2 μm (laser diffraction scattering method), available from Merck KGaA), 3.5 parts by mass of talc (available from Muramatsu Sangyo Co, ltd.), 2.6 parts by mass of triethyl citrate (available from San-Ei Gen F.F.I., Inc.), and 66.6 parts by mass of purified water were mixed to prepare a coating solution.

500 g of the granule obtained in (1) above was coated with the coating solution using a fluidized bed coating device (SFP-01, available from Powrex Corporation) to 2.5 mass % with respect to the granule. The granule was packaged by aluminum heat sealing in an amount of 546.7 mg to obtain a packaged granule containing 200 mg of ibuprofen. The granule of Example 4 contains 7.0 mg of a film-forming polymer and 0.89 mg of a flaky substance in 13.3 mg of the film in one packet.

### Comparative Example 2

26.3 parts by mass of an ammonioalkyl methacrylate copolymer dispersion (solid content 30 mass %, available from Evonik Industries AG), 4.5 parts by mass of talc (available from Muramatsu Sangyo Co, ltd.), 2.6 parts by mass of triethyl citrate (available from San-Ei Gen F.F.I., Inc.), and 66.6 parts by mass of purified water were mixed to prepare a coating solution.

500 g of the granule produced in Example 4 (1) was coated with the coating solution using a fluidized bed coating device (SFP-01, available from Powrex Corporation) to 2.5 mass % with respect to the granule. The granule was packaged by aluminum heat sealing in an amount of 546.7 mg to obtain a packaged granule containing 200 mg of ibuprofen. The granule of Comparative Example 2 contains 7.0 mg of a film-forming polymer in 13.3 mg of the film in one packet but is free from flaky substances.

### Test Example 3: Test for Masking Unpleasant Taste

For the packaged granules of Example 4 and Comparative Example 2, the unpleasant taste when one packet was in the mouth for 15 seconds was evaluated according to the following evaluation criteria by 5 test subjects to determine the average score. Table 5 shows the results.

<Evaluation Criteria for Masking Unpleasant Taste>
0 points: No bitterness or astringency perceived
1 point: Bitterness or astringency slightly perceived
2 points: Bitterness or astringency perceived
3 points: Bitterness or astringency strongly perceived

TABLE 5

| | |
|---|---|
| | Comparative |
| Example 4 | Example 2 |

| | | | | |
|---|---|---|---|---|
| Coating amount | 13.3 | mg/tablet | 13.3 | mg/tablet |
| Film-forming polymer | 7.0 | mg/tablet | 7.0 | mg/tablet |

| | | | |
|---|---|---|---|
| Flaky substance | 0.89 | mg/tablet | — |

| | | |
|---|---|---|
| Test subject 1 | 0 | 1 |
| Test subject 2 | 0 | 2 |
| Test subject 3 | 0 | 2 |
| Test subject 4 | 0 | 2 |
| Test subject 5 | 0 | 2 |
| Average | 0.0 | 1.8 |

As shown in Table 5, the coated solid pharmaceutical preparation with a film containing a flaky substance in addition to the film-forming polymer applied (Example 4) made it difficult to perceive the unpleasant taste derived from the pharmacologically active substance.

### Example 5-1 to Example 5-14

15 g of a film-forming polymer (polyvinyl alcohol-acrylic acid-methyl methacrylate copolymer: POVACOAT, available from Nisshin Kasei Co., Ltd.) and 5 g of the flaky substance shown in Table 6 were dissolved and suspended in 375 g of purified water, to prepare a film-coating solution.

350 g of the uncoated tablet produced in Example 1 (1) was coated with the film-coating solution using a coating machine (HICOATER HC-LABO, available from Freund Corporation) to a mass gain per uncoated tablet of 5 mg to obtain the coated solid pharmaceutical preparation of the disclosure as a film-coated tablet.

TABLE 6

| | Flaky substance composition |
|---|---|
| | (particle size:laser diffraction |
| | scattering method, by Merck KGaA) |
| Example 5-1 | Titanium oxide (50.4%)-coated potassium |
| | aluminum silicate:(49.6%):average |
| | particle size 19.8 μm |
| Example 5-2 | Titanium oxide (53.0%)-coated potassium |
| | aluminum silicate:(47.0%):average |
| | particle size 21.6 μm |
| Example 5-3 | Titanium oxide (46.9%) iron |
| | oxide (9.6%)-coated potassium |
| | aluminum silicate:(43.5%):average |
| | particle size 11.5 μm |
| Example 5-4 | Titanium oxide (39.0%)-coated potassium |
| | aluminum silicate:(61.0%):average |
| | particle size 21.7 μm |
| Example 5-5 | Titanium oxide (36.7%) iron |
| | oxide (3.6%)-coated potassium |
| | aluminum silicate:(59.7%):average |
| | particle size 21.7 μm |
| Example 5-6 | Titanium oxide (40.6%)-coated potassium |
| | aluminum silicate:(59.4%):average |
| | particle size 9.9 μm |
| Example 5-7 | Titanium oxide (41.1%)-coated potassium |
| | aluminum silicate:(58.9%):average |
| | particle size 7.0 μm |
| Example 5-8 | Titanium oxide (48.2%)-coated potassium |
| | aluminum silicate:(51.8%):average |
| | particle size 22.3 μm |
| Example 5-9 | Titanium oxide (36.7%)-coated potassium |
| | aluminum silicate:(63.3%):average |
| | particle size 20.9 μm |
| Example 5-10 | Titanium oxide (46.4%)-coated potassium |
| | aluminum silicate:(53.6%):average |
| | particle size 21.8 μm |
| Example 5-11 | Titanium oxide (27.8%)-coated potassium |
| | aluminum silicate:(72.2%):average |
| | particle size 19.5 μm |
| Example 5-12 | Titanium oxide (43.4%)-coated potassium |
| | aluminum silicate:(56.6%):average |
| | particle size 21.6 μm |
| Example 5-13 | Titanium oxide (37.4%) iron |
| | oxide (5.9%)-coated potassium |
| | aluminum silicate:(56.7%):average |
| | particle size 22.4 μm |
| Example 5-14 | Titanium oxide (12.3%)-coated potassium |
| | aluminum silicate:(87.7%):average |
| | particle size 77.2 μm |

As a result of a test for the deodorant effect in the same manner as in Test Example 1, any of the coated solid pharmaceutical preparations of Example 5-1 to Example 5-14 had an average score of the deodorant effect of 0, which was very excellent.

1. A coated solid pharmaceutical preparation, wherein:
a solid pharmaceutical preparation comprising a pharmacologically active substance is coated with a film comprising a film-forming polymer and a flaky substance.

2. The coated solid pharmaceutical preparation according to claim 1, wherein:
the pharmacologically active substance is one or more selected from the group consisting of ascorbic acid, aspirin, acetaminophen, ibuprofen, ethenzamide, epinastine, ephedrines, caffeine, Chinese herbal extracts, chlorpheniramine, chondroitin sulfate, Na chondroitin sulfate, cysteine, methionine, diphenhydramine, crude drug extracts, theophylline, tranexamic acid, noscapine, Ca pantothenate, vitamin B1 and derivatives thereof, vitamin C, pyridoxine and salts thereof, pirenzepine hydrochloride, fexofenadine, and meloxicam.

3. The coated solid pharmaceutical preparation according to claim 1, wherein:
the pharmacologically active substance is one or more selected from the group consisting of ascorbic acid (Vitamin C), aspirin, acetaminophen, ibuprofen, metamizole, caffeine, chlorpheniramine, diphenhydramine hydrochloride, Vitamin B1 and derivatives thereof.

4. The coated solid pharmaceutical preparation according to claim 1, wherein:
the film-forming polymer is one or more selected from the group consisting of water-soluble polymers, water-insoluble polymers, gastric-soluble polymers, and enteric-soluble polymers.

5. The coated solid pharmaceutical preparation according to claim 4, wherein:
the film-forming polymer is a water-soluble polymer or a water-insoluble polymer.

6. The coated solid pharmaceutical preparation according to claim 1, wherein:
the flaky substance is one or more selected from the group consisting of potassium aluminum silicate, fine silicon dioxide, aluminum silicate, and magnesium aluminum silicate.

7. The coated solid pharmaceutical preparation according to claim 6, wherein:
the flaky substance is one or more selected from the group consisting of potassium aluminum silicate and fine silicon dioxide.

8. The coated solid pharmaceutical preparation according to claim 1, wherein:
the flaky substance is coated with a coloring pigment.

9. The coated solid pharmaceutical preparation according to claim 1, wherein:
a content mass ratio of the flaky substance in the film to the film-forming polymer in the film, [(flaky substance)/(film-forming polymer)], is 0.0002 or more and 20 or less.

10. The coated solid pharmaceutical preparation according to claim 1, wherein:
a mass ratio of the coating amount of the film to the solid pharmaceutical preparation, [(specific film)/(solid pharmaceutical preparation)], is 0.0001 or more and 1 or less.

11. A method for suppressing unpleasant taste and/or unpleasant odor derived from a pharmacologically active substance when a solid pharmaceutical preparation comprising the pharmacologically active substance is taken, the method comprising:
applying a film comprising a film-forming polymer and a flaky substance to the solid pharmaceutical preparation.

12. A method for suppressing discoloration over time when a solid pharmaceutical preparation comprising a pharmacologically active substance is stored, the method comprising:
applying a film comprising a film-forming polymer and a flaky substance to the solid pharmaceutical preparation.

13. The method according to claim 11, wherein the pharmacologically active substance is one or more selected from the group consisting of ascorbic acid (Vitamin C), aspirin, acetaminophen, ibuprofen, metamizole, caffeine, chlorpheniramine, diphenhydramine hydrochloride, Vitamin B1 and derivatives thereof.

14. The coated solid pharmaceutical preparation according to claim 11, wherein:
the flaky substance is one or more selected from the group consisting of potassium aluminum silicate, fine silicon dioxide, aluminum silicate, and magnesium aluminum silicate, and wherein optionally the flaky substance is coasted with a coloring pigment.

15. The method according to claim 14, wherein:
the flaky substance is one or more selected from the group consisting of potassium aluminum silicate and fine silicon dioxide.

16. The method according to claim 12, wherein the pharmacologically active substance is one or more selected from the group consisting of ascorbic acid (Vitamin C), aspirin, acetaminophen, ibuprofen, metamizole, caffeine, chlorpheniramine, diphenhydramine hydrochloride, Vitamin B1 and derivatives thereof.

17. The coated solid pharmaceutical preparation according to claim 12, wherein:
the flaky substance is one or more selected from the group consisting of potassium aluminum silicate, fine silicon dioxide, aluminum silicate, and magnesium aluminum silicate, and wherein optionally the flaky substance is coasted with a coloring pigment.

18. The method according to claim 17, wherein:
the flaky substance is one or more selected from the group consisting of potassium aluminum silicate and fine silicon dioxide.
